(19) United States
(12) Patent Application Publication
(10) Pub. No.: US 2009/0234144 A1
(43) Pub. Date: Sep. 17, 2009

(54) Reactor System and Process for the Manufacture of Ethylene Oxide

(76) Inventors: Alouisius Nicolaas Renee Bos, Amsterdam (NL); Leslie Andew Chewter, Amsterdam (NL); Jeffrey Michael Kobe, Katy, TX (US)

Correspondence Address: SHELL OIL COMPANY, P O BOX 2463, HOUSTON, TX, 772522463, US

(21) Appl. No.: 11/886,800

(22) PCT Filed: Mar. 20, 2006
(86) PCT No.: PCT/US06/09929

§ 371 Date: Jan. 20, 2009

Related U.S. Application Data
(60) Provisional application No. 60/663,984, filed on Mar. 22, 2005.

(51) Int. Cl. C07D 301/10; B01J 8/06

(52) U.S. Cl. 549/534; 422/211; 422/202

(57) ABSTRACT

A reactor system for the epoxidation of ethylene, which reactor system comprises an elongated tube having an internal tube diameter of more than 40 mm, wherein contained is a catalyst bed of catalyst particles comprising silver and a promoter component deposited on a carrier, which promoter component comprises an element selected from rhenium, tungsten, molybdenum and chromium; a process for the epoxidation of ethylene comprising reacting ethylene with oxygen in the presence of the catalyst bed contained in the reactor system; and a method of preparing ethylene glycol, an ethylene glycol ether or an ethanol amine comprising obtaining ethylene oxide by the process for the epoxidation of ethylene, and converting the ethylene oxide into ethylene glycol, the ethylene glycol ether, or the ethanol amine. Preferably, the internal tube diameter is at least 45 mm.

### FIELD OF THE INVENTION

The invention relates to a reactor system. The invention also relates to the use of the reactor system in the manufacture of ethylene oxide, and chemicals derivable from ethylene oxide.

### BACKGROUND OF THE INVENTION

Ethylene oxide is an important industrial chemical used as a feedstock for making such chemicals as ethylene glycol, ethylene glycol ethers, ethanol amines and detergents. One method for manufacturing ethylene oxide is by epoxidation of ethylene, that is the catalyzed partial oxidation of ethylene with oxygen yielding ethylene oxide. The ethylene oxide so manufactured may be reacted with water, an alcohol or an amine to produce ethylene glycol, an ethylene glycol ether or an ethanol amine.

In ethylene epoxidation, a feedstream containing ethylene and oxygen is passed over a bed of catalyst contained within a reaction zone that is maintained at certain reaction conditions. The relatively large heat of reaction makes adiabatic operation at reasonable operation rates impossible. Whilst some of the generated heat may leave the reaction zone as sensible heat, most of the heat needs to be removed through the use of a coolant. The temperature of the catalyst needs to be controlled carefully as the relative rates of epoxidation and combustion to carbon dioxide and water are highly temperature dependent. The temperature dependency together with the relatively large heat of reaction can easily lead to run-away reactions.

A commercial ethylene epoxidation reactor is generally in the form of a shell-and-tube heat exchanger, in which a plurality of substantially parallel elongated, relatively narrow tubes are filled with catalyst particles to form a packed bed, and in which the shell contains a coolant. Irrespective of the type of epoxidation catalyst used, in commercial operation the internal tube diameter is frequently in the range of from 20 to 40 mm, and the number of tubes per reactor may range in the thousands, for example up to 12,000. Reference is made to U.S. Pat. No. 4,921,681, which is incorporated herein by reference.

With the catalyst bed present in narrow tubes, axial temperature gradients over the catalyst bed and hot spots are practically eliminated. In this way, careful control of the temperature of the catalyst is achieved and conditions leading to run-away reactions are substantially avoided.

The large number of the tubes and the narrowness of the tubes represent several difficulties. The commercial reactors are expensive in their manufacture. Also, the filling of the tubes with catalyst particles is time consuming and the catalyst load should be distributed over the many tubes such that all tubes provide the same resistivity under flow conditions.

It would be of a considerable advantage if the catalyst load could be distributed over a smaller number of tubes without compromising the heat and temperature control of the catalyst beds in the reactor.

### SUMMARY OF THE INVENTION

The present invention provides a reactor system for the epoxidation of ethylene, which reactor system comprises at least one elongated tube having an internal tube diameter of more than 40 mm, wherein contained is a catalyst bed of catalyst particles comprising silver and a promoter component deposited on a carrier, which promoter component comprises an element selected from rhenium, tungsten, molybdenum and chromium. More preferably, the internal tube diameter is at least 45 mm.

The invention also provides a process for the epoxidation of ethylene comprising reacting ethylene with oxygen in the presence of the catalyst bed contained in the reactor system of this invention.

Further, the invention provides a method of preparing ethylene glycol, an ethylene glycol ether or an ethanol amine comprising obtaining ethylene oxide by the process for the epoxidation of ethylene according to this invention, and converting the ethylene oxide into ethylene glycol, the ethylene glycol ether, or the ethanol amine.

### DESCRIPTION OF THE DRAWINGS

FIG. 1 depicts an elongated tube which comprises a catalyst bed in accordance with this invention.

FIG. 2 depicts a catalyst particle which may be used in this invention and which has a hollow cylinder geometric configuration.

FIG. 3 is a schematic representation of an ethylene oxide manufacturing process which includes certain novel aspects of the invention.

### DETAILED DESCRIPTION OF THE INVENTION

In accordance with this invention a reactor system is provided which comprises elongated tubes of more than 40 mm, preferably at least 45 mm, and typically up to 80 mm internal tube diameter, which is larger than the conventionally practiced elongated tubes having typically a 20-40 mm internal tube diameter. Increasing the internal tube diameter from, for example, 39 mm to, for example, 55 mm will cause that the number of tubes is approximately halved when the same catalyst load is to be distributed over the tubes applying the same bed depth. Using larger internal tube diameters also allows for the use of larger catalyst particles in the catalyst bed which can lower the pressure drop over the catalyst bed.

Epoxidation catalysts which comprise silver in quantities below 150 g/kg catalyst and additionally a promoter component selected from rhenium, tungsten, molybdenum and chromium have been used commercially for many years. An important aspect of this invention is the recognition only after such many years of commercial use that these catalysts may be used in a reactor tube having an internal tube diameter which is larger than conventionally used, without compromising the temperature and heat control of the catalyst bed. Particularly advantageous is the use of such epoxidation catalysts having silver in quantities of at least 150 g/kg catalyst.

Without wishing to be bound by theory, an important factor may be that these catalysts are less likely to cause a run-away reaction than catalysts which do not comprise a promoter component. Namely, under practical epoxidation conditions, that is in the presence of an organic halide reaction modifier, catalysts which comprise a promoter component produce less heat per mole ethylene converted, and lower activation energies may cause the overall reaction rate to be less temperature dependent. Also, a difference may exist in the catalysts' response to an organic halide: in the case of the catalysts which comprise a promoter component an inadvertent increase in temperature may cause less increase in reaction rate than would be expected just from the temperature increase, and in the case of the catalysts not comprising a promoter component an inadvertent increase in temperature may cause more increase in reaction rate than would be expected just from the temperature increase. Thus, the catalysts' response to the organic halide may have a dampening effect in the case of catalysts which have a promoter component, as opposed to an amplifying effect in the case of catalysts not having a promoter component. The response of the catalysts to an organic halide reaction modifier is known from EP-A-352850, which is incorporated herein by reference.

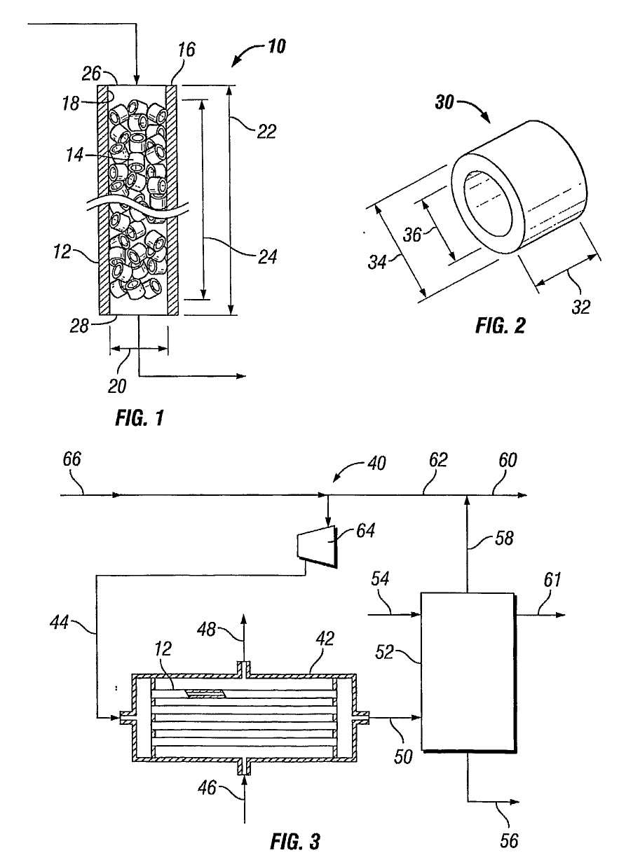

Reference is made to FIG. 1, which depicts the inventive reactor system 10 comprising the elongated tube 12 and the catalyst bed 14, typically a packed catalyst bed, contained within the elongated tube 12. Elongated tube 12 has a tube wall 16 with an inside tube surface 18 and internal tube diameter 20 that define a reaction zone, wherein is contained catalyst bed 14, and a reaction zone diameter 20. Elongated tube 12 has a tube length 22 and the catalyst bed 14 contained within the reaction zone has a bed depth 24.

The internal tube diameter 20 is above 40 mm, preferably 45 mm or above, and typically at most 80 mm. In particular, the internal tube diameter 20 is at least 48 mm, more in particular at least 50 mm. Preferably the internal tube diameter is less than 70 mm, more preferably less than 60 mm. Preferably, the length 22 of the elongated tube is at least 3 m, more preferably at least 5 m. Preferably the tube length 22 is at most 25 m, more preferably at most 20 m. Preferably, the wall thickness of the elongated tube is at least 0.5 mm, more preferably at least 0.8 mm, and in particular at least 1 mm. Preferably, the wall thickness of the elongated tube is at most 10 mm, more preferably at most 8 mm, and in particular at most 5 mm.

Outside the bed depth 24, the elongated tube 12 may contain a separate bed of particles of a non-catalytic or inert material for the purpose of, for example, heat exchange with a feedstream and/or another such separate bed for the purpose of, for example, heat exchange with the reaction product. Preferably, the bed depth 24 is at least 3 m, more preferably at least 5 m. Preferably the bed depth 24 is at most 25 m, more preferably at most 20 m. The elongated tube 12 further has an inlet tube end 26 into which a feedstream comprising ethylene and oxygen can be introduced and an outlet tube end 28 from which a reaction product comprising ethylene oxide and ethylene can be withdrawn. It is noted that the ethylene in the reaction product, if any, is ethylene of the feedstream which passes through the reactor zone unconverted. Typical conversions of the ethylene exceed 10 mole percent, but, in some instances, the conversion may be less.

The reactor system includes a catalyst bed of particles of a catalyst comprising silver and a promoter component deposited on a carrier. In the normal practice of this invention, a major portion of the catalyst bed comprises the catalyst particles. By “a major portion” it is meant that the ratio of the weight of the catalyst particles to the weight of all the particles contained in the catalyst bed, is at least 0.50, in particular at least 0.8, but preferably at least 0.85 and, most preferably at least 0.9. Particles which may be contained in the catalyst bed other than the catalyst particles are, for example, inert particles. However, it is preferred that such other particles are not present.

The carrier for use in this invention may be based on a wide range of materials. Such materials may be natural or artificial inorganic materials and they may include refractory materials, silicon carbide, clays, zeolites, charcoal and alkaline earth metal carbonates, for example calcium carbonate. Preferred are refractory materials, such as alumina, magnesia, zirconia and silica. The most preferred material is α-alumina. Typically, the carrier comprises at least 85% w, more typically at least 90% w, in particular at least 95%/w α-alumina, frequently up to 99.9%/w α-alumina, relative to the weight of the carrier. Other components of the α-alumina carrier may comprise, for example, silica, alkali metal components, for example sodium and/or potassium components, and/or alkaline earth metal components, for example calcium and/or magnesium components.

The surface area of the carrier may suitably be at least 0.1 m²/g, preferably at least 0.3 m²/g, more preferably at least 0.5 m²/g, and in particular at least 0.6 m²/g, relative to the weight of the carrier; and the surface area may suitably be at most 10 m²/g, preferably at most 5 m²/g, and in particular at most 3 m²/g, relative to the weight of the carrier. “Surface area” as used herein is understood to relate to the surface area as determined by the B.E.T. (Brunauer, Emmett and Teller) method as described in Journal of the American Chemical Society 60 (1938) pp. 309-316. High surface area carriers, in particular when they are α-alumina carriers optionally comprising in addition silica, alkali metal and/or alkaline earth metal components, provide improved performance and stability of operation.

The water absorption of the carrier is typically in the range of from 0.2 to 0.8 g/g, preferably in the range of from 0.3 to 0.7 g/g. A higher water absorption may be in favor in view of a more efficient deposition of silver and further elements, if any, on the carrier by impregnation. However, at a higher water absorption, the carrier, or the catalyst made therefrom, may have lower crush strength. As used herein, water absorption is deemed to have been measured in accordance with ASTM C20, and water absorption is expressed as the weight of the water that can be absorbed into the pores of the carrier, relative to the weight of the carrier.

The carrier is typically a calcined, i.e. sintered, carrier, preferably in the form of formed bodies, the size of which is in general determined by the internal diameter of the elongated tube in which the catalyst particles are included in the catalyst bed. In general, the skilled person will be able to determine an appropriate size of the formed bodies. It is found very convenient to use formed bodies in the form of trapezoidal bodies, cylinders, saddles, spheres, doughnuts, and the like. The catalyst particles have preferably a generally hollow cylinder geometric configuration. With reference to FIG. 2, the catalyst particles having a generally hollow cylinder geometric configuration 30 may have a length 32, typically from 4 to 20 mm, more typically from 5 to 15 mm; an outside diameter 34, typically from 4 to 20 mm, more typically from 5 to 15 mm; and inside diameter 36, typically from 0.1 to 6 mm, preferably from 0.2 to 4 mm. Suitably the catalyst particles have a length and an inner diameter as described hereinbefore and an outside diameter of at least 7 mm, preferably at least 8 mm, more preferably at least 9 mm, and at most 20 mm, or at most 15 mm. The ratio of the length 32 to the outside diameter 34 is typically in the range of from 0.5 to 2, more typically from 0.8 to 1.2. While not wanting to be bound to any particular theory, it is believed, however, that the void space provided by the inside diameter of the hollow cylinder allows, when preparing the catalyst, for improved deposition of the catalytic component onto the carrier, for example by impregnation, and improved further handling, such as drying, and, when using the catalyst, it provides for a lower pressure drop over the catalyst bed. An advantage of applying a relatively small bore diameter is also that the shaped carrier material has higher crush strength relative to a carrier material having a larger bore diameter.

In some embodiments, in particular when an α-alumina based carrier is employed, it may be useful for the purpose of improving the selectivity of the catalyst, to coat the carrier surface with tin or a tin compound. Suitably, the quantity of tin may be in the range of from 0.1 to 10% w, more suitable from 0.5 to 5% w, in particular from 1 to 3% w, for example 2% w, calculated as metallic tin relative to the weight of the carrier. Such coating may be applied irrespective of whether or not the carrier will be used for preparing a catalyst comprising the promoter compound. Such coated carriers are known from U.S. Pat. Nos. 4,701,347, 4,548,921 and 3,819,537, which are incorporated herein by reference. The coated carriers may suitably be prepared by impregnating the carrier with a solution of an organic tin compound in an organic diluent, for example toluene or hexane. A suitable organic tin compound may be for example a tin alkoxide or a tin alkanoate. A preferred tin alkanoate is for example tin neodecanoate or tin hexadecanoate. The tin impregnated carrier may be dried in air at a temperature between 400 and 1200° C., for example at 600° C.

The preparation of the catalyst is known in the art and the known methods are applicable to the preparation of the catalyst particles which may be used in the practice of this invention. Methods of depositing silver on the carrier include impregnating the carrier with a silver compound containing cationic silver and performing a reduction to form metallic silver particles. Reference may be made, for example, to U.S. Pat. Nos. 5,380,697, 5,739,075, EP-A-266015, and U.S. Pat. No. 6,368,998, which US patents are incorporated herein by reference.

The reduction of cationic silver to metallic silver may be accomplished during a step in which the catalyst is dried, so that the reduction as such does not require a separate process step. This may be the case if the silver containing impregnation solution comprises a reducing agent, for example, an oxalate, a lactate or formaldehyde.

Appreciable catalytic activity is obtained by employing a silver content of the catalyst of at least 10 g/kg, relative to the weight of the catalyst. Preferably, the catalyst comprises silver in a quantity of from 50 to 500 g/kg, more preferably from 100 to 400 g/kg.

In an embodiment, it is preferred to use catalysts having a high silver content. Preferably, the silver content of the catalyst may be at least 150 g/kg, more preferably at least 200 g/kg, and most preferably at least 250 g/kg, relative to the weight of the catalyst. Preferably, the silver content of the catalyst may be at most 500 g/kg, more preferably at most 450 g/kg, and most preferably at most 400 g/kg, relative to the weight of the catalyst. Preferably, the silver content of the catalyst is in the range of from 150 to 500 g/kg, more preferably from 200 to 400 g/kg, relative to the weight of the catalyst. For example, the catalyst may comprise silver in a quantity of 150 g/kg, or 180 g/kg, or 190 g/kg, or 200 g/kg, or 250 g/kg, or 350 g/kg, relative to the weight of the catalyst. In the preparation of a catalyst having a relatively high silver content, for example in the range of from 150 to 500 g/kg, on total catalyst, it may be advantageous to apply multiple depositions of silver.

The catalyst for use in this invention comprises a promoter component which comprises an element selected from rhenium, tungsten, molybdenum, chromium, and mixtures thereof. Preferably the promoter component comprises, as an element, rhenium.

The promoter component may typically be present in a quantity of at least 0.01 mmole/kg, more typically at least 0.1 mmole/kg, and preferably at least 0.5 mmole/kg, calculated as the total quantity of the element (that is rhenium, tungsten, molybdenum and/or chromium) relative to the weight of the catalyst. The promoter component may be present in a quantity of at most 50 mmole/kg, preferably at most 10 mmole/kg, more preferably at most 5 mmole/kg, calculated as the total quantity of the element relative to the weight of the catalyst. The form in which the promoter component may be deposited onto the carrier is not material to the invention. For example, the promoter component may suitably be provided as an oxide or as an oxyanion, for example, as a rhenate, perrhenate, or tungstate, in salt or acid form.

When the catalyst comprises a rhenium containing promoter component, rhenium may typically be present in a quantity of at least 0.1 mmole/kg, more typically at least 0.5 mmole/kg, and preferably at least 1.0 mmole/kg, in particular at least 1.5 mmole/kg, calculated as the quantity of the element relative to the weight of the catalyst. Rhenium is typically present in a quantity of at most 5.0 mmole/kg, preferably at most 3.0 mmole/kg, more preferably at most 2.0 mmole/kg, in particular at most 1.5 mmole/kg.

Further, when the catalyst comprises a rhenium containing promoter component, the catalyst may preferably comprise a rhenium copromoter, as a further component deposited on the carrier. Suitably, the rhenium copromoter may be selected from components comprising an element selected from tungsten, chromium, molybdenum, sulfur, phosphorus, boron, and mixtures thereof. Preferably, the rhenium copromoter is selected from components comprising tungsten, chromium, molybdenum, sulfur, and mixtures thereof. It is particularly preferred that the rhenium copromoter comprises, as an element, tungsten.

The rhenium copromoter may typically be present in a total quantity of at least 0.01 mmole/kg, more typically at least 0.1 mmole/kg, and preferably at least 0.5 mmole/kg, calculated as the element (i.e. the total of tungsten, chromium, molybdenum, sulfur, phosphorus and/or boron), relative to the weight of the catalyst. The rhenium copromoter may be present in a total quantity of at most 40 mmole/kg, preferably at most 10 mmole/kg, more preferably at most 5 mmole/kg, on the same basis. The form in which the rhenium copromoter may be deposited on the carrier is not material to the invention. For example, it may suitably be provided as an oxide or as an oxyanion, for example, as a sulfate, borate or molybdate, in salt or acid form.

The catalyst preferably comprises silver, the promoter component, and a component comprising a further element, deposited on the carrier. Eligible further elements may be selected from the group of nitrogen, fluorine, alkali metals, alkaline earth metals, titanium, hafnium, zirconium, vanadium, thallium, thorium, tantalum, niobium, gallium and germanium and mixtures thereof. Preferably the alkali metals are selected from lithium, potassium, rubidium and cesium. Most preferably the alkali metal is lithium, potassium and/or cesium. Preferably the alkaline earth metals are selected from calcium and barium. Typically, the further element is present in the catalyst in a total quantity of from 0.01 to 500 mmole/kg, more typically from 0.05 to 100 mmole/kg, calculated as the element on the weight of the catalyst. The further elements may be provided in any form. For example, salts of an alkali metal or an alkaline earth metal are suitable.

As used herein, the quantity of alkali metal present in the catalyst is deemed to be the quantity insofar as it can be extracted from the catalyst with de-ionized water at 100° C. The extraction method involves extracting a 10-gram sample of the catalyst three times by heating it in 20 ml portions of de-ionized water for 5 minutes at 100° C. and determining in the combined extracts the relevant metals by using a known method, for example atomic absorption spectroscopy.

As used herein, the quantity of alkaline earth metal present in the catalyst is deemed to the quantity insofar as it can be extracted from the catalyst with 10% w nitric acid in de-ionized water at 100° C. The extraction method involves extracting a 10-gram sample of the catalyst by boiling it with a 100 ml portion of 10% w nitric acid for 30 minutes (1 atm., i.e. 101.3 kPa) and determining in the combined extracts the relevant metals by using a known method, for example atomic absorption spectroscopy. Reference is made to U.S. Pat. No. 5,801,259, which is incorporated herein by reference.

A catalyst which may suitably be used in this invention is a catalyst designated S-882, as has been marketed by CRI International (Houston, Tex., USA).

FIG. 3 is a schematic representation showing a typical ethylene oxide manufacturing system 40 with a shell-and-tube heat exchanger 42 which is equipped with one or more reactor systems as depicted in FIG. 1. Typically a plurality of reactor systems of this invention is grouped together into a tube bundle for insertion into the shell of a shell-and-tube heat exchanger. The skilled person will understand that the catalyst particles may be packed into the individual elongated tubes such that the elongated tubes and their contents provide the same resistivity when a gas flow passes through the elongated tubes. The number of elongated tubes present in the shell-and-tube heat exchanger 42 is typically in the range of from 1,000 to 15,000, more typically in the range of from 2,000 to 10,000. Generally, such elongated tubes are in a substantially parallel position relative to each other. Ethylene oxide manufacturing system 40 may comprise one or more shell-and-tube heat exchangers 42, for example two, three or four.

In particular for testing purposes, the shell-and-tube heat exchanger 42 may comprise elongated tubes which are individually removable from the shell-and-tube heat exchanger and exchangeable against elongated tubes of a different internal diameter. As an alternative, the elongated tubes may be removable and exchangeable as one or more bundles. If desirable, the performance of the catalyst may be tested in the shell-and-tube heat exchanger having elongated tubes of different internal diameters.

A feedstream comprising ethylene and oxygen is charged via conduit 44 to the tube side of shell-and-tube heat exchanger 42 wherein it is contacted with the catalyst bed contained therein within elongated tubes 12 of the inventive reactor systems. The shell-and-tube heat exchanger 42 is typically operated in a manner which allows an upward or downward flow of gas through the catalyst bed. The heat of reaction is removed and control of the reaction temperature, that is the temperature within the catalyst bed, is achieved by use of a heat transfer fluid, for example oil, kerosene or water, which is charged to the shell side of shell-and-tube heat exchanger 42 by way of conduit 46 and the heat transfer fluid is removed from the shell of shell-and-tube heat exchanger 42 through conduit 48.

The reaction product comprising ethylene oxide, unreacted ethylene, unreacted oxygen and, optionally, other reaction products such as carbon dioxide and water, is withdrawn from the reactor system tubes of shell-and-tube heat exchanger 42 through conduit 50 and passes to separation system 52. Separation system 52 provides for the separation of ethylene oxide from ethylene and, if present, carbon dioxide and water. An extraction fluid such as water can be used to separate these components and is introduced to separation system 52 by way of conduit 54. The enriched extraction fluid containing ethylene oxide passes from separation system 52 through conduit 56 while unreacted ethylene and carbon dioxide, if present, passes from separation system 52 through conduit 58. Separated carbon dioxide passes from separation system 52 through conduit 61. A portion of the gas stream passing through conduit 58 can be removed as a purge stream through conduit 60. The remaining gas stream passes through conduit 62 to recycle compressor 64. A stream containing ethylene and oxygen passes through conduit 66 and is combined with the recycle ethylene that is passed through conduit 62 and the combined stream is passed to recycle compressor 64. Recycle compressor 64 discharges into conduit 44 whereby the discharge stream is charged to the inlet of the tube side of the shell-and-tube heat exchanger 42. Ethylene oxide produced may be recovered from the enriched extraction fluid, for example by distillation or extraction.

The ethylene concentration in the feedstream passing through conduit 44 may be selected within a wide range. Typically, the ethylene concentration in the feedstream will be at most 80 mole-%, relative to the total feed. Preferably, it will be in the range of from 0.5 to 70 mole-%, in particular from 1 to 60 mole-%, on the same basis. As used herein, the feedstream is considered to be the composition which is contacted with the catalyst particles.

The present epoxidation process may be air-based or oxygen-based, see “Kirk-Othmer Encyclopedia of Chemical Technology”, 3^{rd} edition, Volume 9, 1980, pp. 445-447. In the air-based process air or air enriched with oxygen is employed as the source of the oxidizing agent while in the oxygen-based processes high-purity (at least 95 mole-%) oxygen is employed as the source of the oxidizing agent. Presently most epoxidation plants are oxygen-based and this is a preferred embodiment of the present invention.

The oxygen concentration in the feedstream passing through conduit 44 may be selected within a wide range. However, in practice, oxygen is generally applied at a concentration which avoids the flammable regime. Typically, the concentration of oxygen applied will be within the range of from 1 to 15 mole-%, more typically from 2 to 12 mole-% of the total feed. The actual safe operating ranges depend, along with the feedstream composition, also on the reaction conditions such as the reaction temperature and the pressure.

An organic halide may be present in the feedstream passing through conduit 44 as a reaction modifier for increasing the selectivity, suppressing the undesirable oxidation of ethylene or ethylene oxide to carbon dioxide and water, relative to the desired formation of ethylene oxide. Fresh organic halide is suitably fed to the process through conduit 66. Organic halides are in particular organic bromides, and more in particular organic chlorides. Preferred organic halides are chlorohydrocarbons or bromohydrocarbons. More preferably they are selected from the group of methyl chloride, ethyl chloride, ethylene dichloride, ethylene dibromide, vinyl chloride or a mixture thereof. Most preferred are ethyl chloride and ethylene dichloride.

The organic halides are generally effective as reaction modifier when used in low concentration in the feed, for example up to 0.01 mole-%, relative to the total feed. It is preferred that the organic halide is present in the feedstream at a concentration of at most 50×10⁻⁴ mole-%, in particular at most 20×10⁻⁴ mole-%, more in particular at most 15×10⁻⁴ mole-%, relative to the total feed, and preferably at least 0.2×10⁻⁴ mole-%, in particular at least 0.5×10⁻⁴ mole-%, more in particular at least 1×10⁻⁴ mole-%, relative to the total feed.

In addition to ethylene, oxygen and the organic halide, the feedstream may contain one or more optional components, for example carbon dioxide, inert gases and saturated hydrocarbons. Carbon dioxide generally has an adverse effect on the catalyst activity. Advantageously, separation system 52 is operated in such a way that the quantity of carbon dioxide in the feedstream through conduit 44 is low, for example, below 2 mole-%, preferably below 1 mole-%, or in the range of from 0.2 to 1 mole-%. Inert gases, for example nitrogen or argon, may be present in the feedstream passing through conduit 44 in a concentration of from 30 to 90 mole-%, typically from 40 to 80 mole-%. Suitable saturated hydrocarbons are methane and ethane. If saturated hydrocarbons are present, they may be present in a quantity of up to 80 mole-%, relative to the total feed, in particular up to 75 mole-%. Frequently they are present in a quantity of at least 30 mole-%, more frequently at least 40 mole-%. Saturated hydrocarbons may be employed in order to increase the oxygen flammability limit. Olefins other than ethylene may be present in the feedstream, for example in a quantity of less than 10 mole-%, in particular less than 1 mole-%, relative to the quantity of ethylene. However, it is preferred that ethylene is the single olefin present in the feedstream.

The epoxidation process may be carried out using reaction temperatures selected from a wide range. Preferably the reaction temperature is in the range of from 150 to 340° C., more preferably in the range of from 180 to 325° C. Typically, the shell-side heat transfer liquid has a temperature which is typically 1 to 15° C., more typically 2 to 10° C. lower than the reaction temperature.

In order to reduce the effects of deactivation of the catalyst, the reaction temperature may be increased gradually or in a plurality of steps, for example in steps of from 0.1 to 20° C., in particular 0.2 to 10° C., more in particular 0.5 to 5° C. The total increase in the reaction temperature maybe in the range of from 10 to 140° C., more typically from 20 to 100° C. The reaction temperature may be increased typically from a level in the range of from 150 to 300° C., more typically from 200 to 280° C., when a fresh catalyst is used, to a level in the range of from 230 to 340° C., more typically from 240 to 325° C., when the catalyst has decreased in activity due to ageing.

The epoxidation process is preferably carried out at a pressure in the inlet tube end 26 in the range of from 1000 to 3500 kPa. “GHSV” or Gas Hourly Space Velocity is the unit volume of gas at normal temperature and pressure (0° C., 1 atm, i.e. 101.3 kPa) passing over one unit of the total volume of catalyst bed per hour. Preferably, the GHSV is in the range of from 1500 to 10000 Nm³/(m³.h). Preferably, the process is carried out at a work rate in the range of from 0.5 to 10 kmole ethylene oxide produced per m³ of the total catalyst bed per hour, in particular 0.7 to 8 kmole ethylene oxide produced per m³ of the total catalyst bed per hour, for example 5 kmole ethylene oxide produced per m³ of the total catalyst bed per hour.

The ethylene oxide produced in the epoxidation process may be converted, for example, into ethylene glycol, an ethylene glycol ether or an ethanol amine.

The conversion into ethylene glycol or the ethylene glycol ether may comprise, for example, reacting the ethylene oxide with water, suitably using an acidic or a basic catalyst. For example, for making predominantly the ethylene glycol and less ethylene glycol ether, the ethylene oxide may be reacted with a ten fold molar excess of water, in a liquid phase reaction in presence of an acid catalyst, e.g. 0.5-1.0% w sulfuric acid, based on the total reaction mixture, at 50-70° C. at 100 kPa absolute, or in a gas phase reaction at 130-240° C. and 2000-4000 kPa absolute, preferably in the absence of a catalyst. If the proportion of water is lowered the proportion of ethylene glycol ethers in the reaction mixture is increased. The ethylene glycol ethers thus produced may be a di-ether, tri-ether, tetra-ether or a subsequent ether. Alternative ethylene glycol ethers may be prepared by converting the ethylene oxide with an alcohol, in particular a primary alcohol, such as methanol or ethanol, by replacing at least a portion of the water by the alcohol.

The ethylene oxide may be converted into ethylene glycol by first converting the ethylene oxide into ethylene carbonate by reacting it with carbon dioxide, and subsequently hydrolyzing the ethylene carbonate to form ethylene glycol. For applicable methods, reference is made to U.S. Pat. No. 6,080,897, which is incorporated herein by reference.

The conversion into the ethanol amine may comprise reacting ethylene oxide with an amine, such as ammonia, an alkyl amine or a dialkyl amine. Anhydrous or aqueous ammonia may be used. Anhydrous ammonia is typically used to favor the production of mono ethanol amine. For methods applicable in the conversion of ethylene oxide into the ethanol amine, reference may be made to, for example U.S. Pat. No. 4,845,296, which is incorporated herein by reference.

Ethylene glycol and ethylene glycol ethers may be used in a large variety of industrial applications, for example in the fields of food, beverages, tobacco, cosmetics, thermoplastic polymers, curable resin systems, detergents, heat transfer systems, etc. Ethanol amines may be used, for example, in the treating (“sweetening”) of natural gas.

Unless specified otherwise, the organic compounds mentioned herein, for example the olefins, ethylene glycol ethers, ethanol amines and organic halides, have typically at most 40 carbon atoms, more typically at most 20 carbon atoms, in particular at most 10 carbon atoms, more in particular at most 6 carbon atoms. As defined herein, ranges for numbers of carbon atoms (i.e. carbon number) include the numbers specified for the limits of the ranges.

The following examples are intended to illustrate the advantages of the present invention and are not intended to unduly limit the scope of the invention.

### EXAMPLE I

### Comparative, Not According to the Invention

Reactor models were developed which include appropriate kinetic models for the use of silver containing catalysts in a process for manufacturing ethylene oxide from ethylene and oxygen. An appropriate reactor model was developed for silver catalysts comprising rhenium and tungsten and another appropriate reactor model was developed for silver catalysts containing no rhenium and no rhenium copromoter.

The models are based on the correlation of actual catalyst performance data gathered from numerous sources such as micro-reactor activity data, pilot plant data and other sources of catalyst performance data.

Using the appropriate reactor model a process was modeled, as performed in a reactor tube of 11.8 m length and 38.9 mm internal diameter containing a packed bed of standard cylindrical catalyst particles having about 8 mm outside diameter 34, about 8 mm length 32 and about 3.2 mm inside diameter 36, the catalyst comprising silver, rhenium, and tungsten, and the reactor tube being cooled in a boiling water reactor. The quantity of silver was 275 g/kg, relative to the weight of the catalyst. The operating conditions of the modeled process were a GHSV of 3327 Nl/l.h, inlet pressure of 1.75 MPa, a work rate of 3.3 kmole ethylene oxide per m³ of packed bed per hour, and a composition of the feed stream of 25 mole-% ethylene, 8.5 mole-% oxygen, 1 mole-% carbon dioxide, 1 mole-% nitrogen, 2.7 mole-% argon, 1 mole-% ethane, the balance being methane. The selectivity of the catalyst is estimated to be 89.9 mole-%.

The shell-side coolant temperature was calculated to be 230° C. The model predicted that in a tube of this internal diameter (38.9 mm) the coolant temperature can be increased to 247° C. before the rate of production of reaction heat exceeds the rate of heat removal through the wall of the tube, which is characteristic of a run-away reaction. Thus, according to the model prediction, under these conditions the margin to run-away is 17° C.

### EXAMPLE II

Example I was repeated, with the difference that the internal diameter was 54.4 mm, instead of 38.9 mm.

The shell-side coolant temperature was calculated to be 228° C. The model predicted that in a tube of this internal diameter (54.4 mm) the coolant temperature can be increased to 240° C. before the rate of production of reaction heat exceeds the rate of heat removal through the wall of the tube. Thus, according to the model prediction, under these conditions the margin to run-away is 12° C.

### EXAMPLE III

### Comparative, Not According to the Invention

Example I was repeated, with the difference that the catalyst comprised silver in a quantity of 132 g/kg, relative to the weight of the catalyst. The selectivity of the catalyst is estimated to be 89.1 mole-%.

The shell-side coolant temperature was calculated to be 234° C. The model predicted that in a tube of this internal diameter (38.9 mm) the coolant temperature can be increased to 247° C. before the rate of production of reaction heat exceeds the rate of heat removal through the wall of the tube. Thus, according to the model prediction, under these conditions the margin to run-away is 13° C.

### EXAMPLE IV

Example III was repeated, with the difference that the internal diameter was 54.4 mm, instead of 38.9 mm.

The shell-side coolant temperature was calculated to be 232° C. The model predicted that in a tube of this internal diameter (54.4 mm) the coolant temperature can be increased to 240° C. before the rate of production of reaction heat exceeds the rate of heat removal through the wall of the tube. Thus, according to the model prediction, under these conditions the margin to run-away is 8° C.

### EXAMPLE V

### Comparative, Not According to the Invention

Example I was repeated, with the differences that the catalyst comprises silver in a quantity of 145 g/kg, relative to the weight of the catalyst, no rhenium and no rhenium copromoter, that the appropriate reactor model for a silver catalyst containing no rhenium and no rhenium copromoter was used, and that the internal diameter was 38.5 mm, instead of 38.9 mm. The selectivity of the catalyst is estimated to be 82.7 mole-%.

The shell-side coolant temperature was calculated to be 199° C. The model predicted that in a tube of this internal diameter (38.5 mm) the coolant temperature can be increased to 209° C. before the rate of production of reaction heat exceeds the rate of heat removal through the wall of the tube. Thus, according to the model prediction, under these conditions the margin to run-away is 10° C.

### EXAMPLE VI

### Comparative, Not According to the Invention

Example V was repeated, with the difference that the internal diameter was 55 mm, instead of 38.5 mm.

The shell-side coolant temperature was calculated to be 194.5° C. The model predicted that in a tube of this internal diameter (55 mm) the coolant temperature can be increased to 197.5° C. before the rate of production of reaction heat exceeds the rate of heat removal through the wall of the tube. Thus, according to the model prediction, under these conditions the margin to run-away is as low as 3° C.

These calculated Examples show that when an epoxidation catalyst containing a promoter component is present in a reactor tube which is wider than conventionally applied, under epoxidation conditions the margin to run-away may be as large as the margin to run-away which is applicable for an epoxidation catalyst not containing the promoter component when present in a reactor tube of conventional diameter. This means that the epoxidation catalyst containing a promoter component can be applied in a reactor tube which is wider than conventionally applied without compromising the temperature and heat control of the catalyst bed.

These calculated Examples also show that when an epoxidation catalyst containing a promoter component and a relatively high silver content is used, irrespective of the internal tube diameter, a larger margin to run-away can be observed than for an epoxidation catalyst containing a promoter component and a lower silver content.

1. A reactor system for the epoxidation of ethylene, which reactor system comprises at least one elongated tube having an internal tube diameter of more than 40 mm, wherein contained is a catalyst bed of catalyst particles comprising silver in a quantity of at least 150 g/kg, relative to the weight of the catalyst, and a promoter component deposited on a carrier, which promoter component comprises an element selected from the group consisting of rhenium, tungsten, molybdenum and chromium.

2. A reactor system as claimed in claim 1, wherein the internal tube diameter is at least 45 mm.

3. A reactor system as claimed in claim 1, wherein the internal tube diameter is in the range of from 45 to 80 mm.

4. A reactor system as claimed in claim 1, wherein the length of the elongated tube is in the range of from 3 to 25 m, and the wall thickness of the elongated tube is in the range of from 0.5 to 10 mm.

5. A reactor system as claimed in claim 1, wherein the elongated tube is contained in a shell-and-tube heat exchanger, and the number of such elongated tubes contained in the shell-and-tube heat exchanger is in the range of from 1,000 to 15,000.

6. A reactor system as claimed in claim 1, wherein the catalyst particles have a generally hollow cylinder geometric configuration having a length of from 4 to 20 mm, an outside diameter of from 4 to 20 mm an inside diameter of from 0.1 to 6 mm and a ratio of the length to the outside diameter in the range of from 0.5 to 2.

7. A reactor system as claimed in claim 1, wherein the catalyst comprises silver, a rhenium containing promoter component, a rhenium copromoter selected from components comprising an element selected from tungsten, chromium, molybdenum, sulfur, phosphorus, boron, and mixtures thereof, deposited on a carrier comprising α-alumina.

8. A reactor system as claimed in claim 1, wherein the catalyst comprises silver in a quantity of at least 200 g/kg, relative to the weight of the catalyst.

9. A reactor system as claimed in claim 1, wherein the catalyst comprises silver in a quantity of from 200 to 400 g/kg, relative to the weight of the catalyst.

10. A process for the epoxidation of ethylene comprising reacting ethylene with oxygen in the presence of the catalyst bed contained in a reactor system as claimed in claim 1.

11. A process as claimed in claim 10, wherein ethylene is reacted with oxygen in the additional presence of one or more organic halides.

12. A process as claimed in claim 11, wherein the one or more organic halides are selected from chlorohydrocarbons and bromohydrocarbons.

13. A method of preparing ethylene glycol, an ethylene glycol ether or an ethanol amine comprising obtaining ethylene oxide by a process for the epoxidation of ethylene as claimed in claim 10, and converting the ethylene oxide into ethylene glycol, the ethylene glycol ether, or the ethanol amine.

14. A reactor system as claimed in claim 1, wherein the internal tube diameter is in the range of from 48 to 70 mm.

15. A reactor system as claimed in claim 1, wherein the internal tube diameter is in the range of from 50 to 60 mm.

16. A reactor system as claimed in claim 2, wherein the length of the elongated tube is in the range of from 5 to 20 m, and the wall thickness of the elongated tube is in the range of from 1 to 5 mm.

17. A reactor system as claimed in claim 1, wherein the catalyst particles have a generally hollow cylinder geometric configuration having a length of from 5 to 15 mm, an outside diameter of from 5 to 15 mm, an inside diameter of from 0.2 to 4 mm; and a ratio of the length to the outside diameter in the range of from 0.8 to 1.2.

18. A reactor system as claimed in claim 17, wherein the catalyst comprises silver, a rhenium containing promoter component, a rhenium copromoter selected from components comprising an element selected from tungsten, chromium, molybdenum, sulfur, phosphorus, boron, and mixtures thereof, deposited on a carrier comprising α-alumina.

19. A reactor system as claimed in claim 7, wherein the catalyst comprises silver in a quantity of at least 200 g/kg, relative to the weight of the catalyst.

20. A reactor system as claimed in claim 18, wherein the catalyst comprises silver in a quantity of at least 200 g/kg, relative to the weight of the catalyst.
